(19) United States
(12) Patent Application Publication
(10) Pub. No.: US 2006/0275172 A1
(43) Pub. Date: Dec. 7, 2006

(54) Item reprocessing and sterile packaging apparatus

(76) Inventor: Terrence R. Langford, Tucson, AZ (US)

Correspondence Address: QUARLES & BRADY STREICH LANG, LLP, ONE SOUTH CHURCH AVENUE, SUITE 1700, TUCSON, AZ, 85701-1621, US

(21) Appl. No.: 11/143,118

(22) Filed: Jun. 2, 2005

(51) Int. Cl. A61L 2/18; A61L 2/20

(52) U.S. Cl. 422/28; 422/292; 53/425

(57) ABSTRACT

Methods and apparatus for reprocessing and packaging items. An item is cleaned within a push/pull reprocessing apparatus, contained within sterilizable packaging, and sterilized within said apparatus. Alternatively, an item is first cleaned and sterilized within a push/pull reprocessing apparatus and then contained within sterile packaging. The apparatus includes one or multiple chambers within which cleaning and/or sterilizing take place and dispenser of sterile or sterilizable packaging.

### BACKGROUND OF THE INVENTION

1. Field of the Invention

This invention relates generally to improved methods for packaging items after treatment by push/pull reprocessing systems used to reprocess soiled items to an endpoint of high-level disinfection or better, and, more particularly, to methods that are especially useful in the reprocessing and sterile packaging of items utilizing a single reprocessing and packaging apparatus.

2. Description of the Related Art

The reprocessing (i.e., cleaning and decontamination) of items that come into contact with the bodily substances of people or animals such that they are substantially “substance free” (of, e.g., viruses, bacteria, detergent, sterilant, lipids, etc.) represent an immense and ongoing challenge. This challenge has been underscored by a recent article entitled “Widely used sterilizer under attack” (published in Jan. 21, 2003 edition of the newspaper USA Today). The article describes a fatal outbreak of bacterial infection that was linked to the improper sterilization of hospital bronchoscopes. Despite the hospital's use of one of the most popular sterilizing systems, tests performed by the Centers for Disease Control and Prevention found bacteria on the system's water filters and in its rinse water. This and other infection outbreaks has led to continuing controversy over how best to clean and sterilize used endoscopes.

The contaminants typically found on tubular or “lumened” medical items, such as endoscopes, are especially difficult to remove. In addition to fecal mater, loose cellular debris, blood and blood products, viruses, and bacteria, an endoscope can be coated with various hydrophobic films, such as “biofilm” material. A biofilm typically comprises cells, both dead and alive, cell debris and extracellular polymer substances. Once biofilm is formed by microorganisms (including bacteria, fungi, and protozoans), these microorganisms can colonize and replicate on the interior surfaces of tubing, forming a protective slime layer known as a “glycocalyx” that is especially difficult to remove.

Merely soaking endoscopes in a sterilant or detergent is unacceptable since numerous pockets exist within the tubing where the sterilant or detergent cannot reach effectively, which leaves areas of contamination within the endoscope. Moreover, with the prevalence of highly contagious diseases such as hepatitis B and C and Acquired Immune Deficiency Syndrome, reliable sterilization or disposal of all used medical tools seemingly becomes mandatory. Yet, while many medical instruments today are routinely cleaned, disinfected, and reused, experts in the field recently have warned that some of the more difficult to clean and sterilize medical items are putting people at risk.

Indeed, one expert has stated that there are no independent published reports or data anywhere in the medical literature that show liquid chemical sterilants (or any other method/process/agent) can be used to reliably “sterilize” flexible endoscopes or other complex, lumened instruments (See Comments by L. Muscarella (Custom Ultrasonics) on AAMI TIR7:1999, Chemical Sterilants and Sterilization Methods: A Guide to Selection and Use, downloaded from the website myendosite.com).

To the contrary, Kovacs et al. reports that a strain of Pseudomonas aeruginosa has been repeatedly isolated from tap water used for cleaning and rinsing endoscopes and appears to be responsible for three separate clinical episodes of endoscopic retrograde cholangio-pancreatography (ERCP)-associated cholangitis over an 11-yr period. These authors also conclude that the organism is resistant to a commonly used sterilant because it was recovered from a variety of endoscopes that had undergone stringent reprocessing protocols (see Kovacs B J, et al. “Efficacy of various disinfectants in killing a resistant strain of Pseudomonas aeruginosa by comparing zones of inhibition: Implications for endoscopic equipment reprocessing,” Am J Gastroenterol 1998;93:2057-9). Thus, there is a genuine need for reliable methods of high-level disinfection or better to help ensure that even chemical-resistant pathogens are effectively eliminated.

Furthermore, some chemical cleaners or sterilants are so harshly reactive that they can damage the items they are meant to clean or sterilize. Thus, the problems encountered during item (and especially medical item) cleaning and disinfecting primarily involve trying to strike a balance between ensuring as much as possible the complete removal of contaminants and chemicals while, at the same time, not damaging the instrument.

After sterilization, endoscopes typically are rinsed with water filtered down to the 0.2 micron (200 nanometer) level. Unfortunately, many viruses, endotoxins, and prions are smaller than 200 nanometers, meaning that they can remain in the water even after filtration. Also, as reported in the articles mentioned above, water and water filters are known sources of contamination. Even more troubling, however, is the statement by one expert that “there are no independent data in the medical literature that support the production of sterile water (a biological endpoint defined as containing fewer than 10⁻⁶ CFU/ml or fewer than 5 endotoxin units/ml) by passing unprocessed water (that is, un-sterilized water, such as water that flows though a hospital's tap) through a bacterial (e.g., 0.1 or 0.2 micron) filtration system” (See Comments by L. Muscarella (Custom Ultrasonics) on AAMI TIR7:1999, Chemical Sterilants and Sterilization Methods: A Guide to Selection and Use, downloaded from the website myendosite.com). Moreover, there is no currently available system that monitors the biological content of filtered water to insure its sterility when used in conjunction with medical item cleaning or sterilization apparatuses. Finally, having to add additional sterilization steps and/or use sterilized (e.g., autoclaved) water becomes tedious and expensive.

So called “push/pull reprocessing systems” are automatic apparatuses that include a chamber containing a baffle with one or more openings through which water (or another fluid) surges back-and-forth (hence “push/pull”) through the opening or openings in the baffle. When soiled items, such as endoscopes and other lumened instruments, are placed within an opening, fluid also surges through them. Accordingly, a back-and-forth “scrubbing action” is created by the surging fluid the contacts any accessible surface on an item, including a lumen or lumens.

Such systems have been previously been described to provide superb cleaning based on physical inspection (for example, U.S. Pat. No. 5,753,195). However, as evidenced by the above media and scientific articles, methods for high-level or greater disinfection of soiled items have been a long sought after yet difficult to attain goal.

Moreover, once an item has undergone reprocessing (including sterilization), it must either be used immediately or packaged under sterile conditions for storage and later use. However, the maintenance of sterile conditions can be problematic if items are packaged outside of the reprocessor.

Therefore, there continues to be a need for a reprocessing method and apparatus that includes the ability to package items while maintaining a sterile environment.

### SUMMARY OF THE INVENTION

The invention generally involves methods and apparatus for reprocessing and packaging items. According to a preferred method of the invention, an item is cleaned within a push/pull reprocessing apparatus, contained within sterilizable packaging, and sterilized within the reprocessing apparatus. Alternatively, an item is first cleaned and sterilized within a push/pull reprocessing apparatus and then contained within sterile packaging while still in the apparatus. The apparatus of the invention includes one or multiple chambers, within which cleaning and/or sterilizing take place, and a dispenser of sterile or sterilizable packaging.

The packaging of the invention may include a disposable sleeve, such as a polyethylene sleeve with sealable ends, various types of treated paper, shrink wrapping, and the like. In one embodiment, the reprocessed item is first sterilized and then lifted and positioned inside a sterile bag, the end of which is then heat sealed, all while contained within the closed, sterile environment of the reprocessor.

In another embodiment of the invention, a method for reprocessing and packaging items includes the steps of cleaning an item within a push/pull reprocessing apparatus, removing the item from said apparatus, containing the item within sterilizable packaging, replacing the contained item into the apparatus, and sterilizing the contained item within the reprocessing apparatus. In this manner, an item may be packaged manually and then sterilized prior to sealing of the packaging.

Thus, it is a primary objective of the invention to provide a medical item reprocessing and packaging method that improves reliably of item sterility, especially when the item must be packaged for later use.

In accordance with the above methods, there is provided new and improved apparatus for reprocessing and packaging an item. The combination reprocessor and packaging apparatus features a push/pull reprocessing system and a dispenser of sterile or sterilizable packaging coupled to the inside or outside of the reprocessor. The apparatus of the invention contains a single or multiple chambers within which cleaning and/or sterilization take place.

Various other purposes and advantages of the invention will become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### DETAILED DESCRIPTION OF THE PREFERRED EMBODIMENTS

The invention relates generally to a methods for reprocessing and packaging items that involve cleaning an item within a push/pull reprocessing apparatus, containing said item within sterilizable packaging, and sterilizing said contained item within said apparatus, these last two steps being reversible in order. The method may further include the step of drying the item prior to the step of containing it within packaging.

The apparatus for reprocessing and packaging an item includes a push/pull reprocessing system and a dispenser of sterilizable packaging coupled to the reprocessor. In a preferred embodiment, the apparatus of the invention has the dispenser disposed upon the interior of the reprocessing apparatus so at to maintain a closed, sterile environment.

The reprocessing apparatus may contain a single chamber within which both cleaning and sterilization takes place or a cleaning chamber and a separate sterilization (before or after packaging) chamber.

The terms “push/pull apparatus,” “push/pull system,” “cleaner/sterilizer,” “push/pull reprocessor,” “reprocessor,” and “reprocessing” as used throughout the specification are meant to be synonymous with the use of a push-pull apparatus (such as the Langford IC Systems, Inc. Manzi Mark 1) that cleans items (e.g., endoscopes, dental appliances, and the like) by surging fluid back-and-forth upon the accessible surfaces of these items.

Preferably, sterile ozonated water is provided during the sterilization step. Because ozone is an unstable molecule, it should be generated at the point of application. Ozone can be produced several ways, although one method, corona discharge, predominates in the ozone generation industry. Ozone can also be produced by irradiating an oxygen-containing gas with ultraviolet light, electrolytic reaction and other emerging technologies. Most ozone generators currently use ultraviolet radiation. These are usually the lowest cost ozone generators on a per unit basis. For additional guidance in ozone production and its uses, see, for example, U.S. Pat. No. 5,207,237.

For methods of the invention involving the use of an ozonated water rinse, it is important to recognize that the time of exposure and concentration of ozone will vary based on a number of parameters, such as the quantity and size of items being treated, and the volume of the cleaning or sterilizing apparatus. Preferably, ozonated water is used to rinse items for 1-10 minutes at a concentration of 1-10% ozone by volume. Moreover, the use of a filter is preferred to keep water free of particles above 10 microns in size and most preferably 0.1 microns or less in size.

Within the following examples, endoscopes or other medical or dental instruments will be used as an example of an item or instrument to be reprocessed. However, the inventor contemplates use of the invention with any tubular item, as well as a variety of other items such as circuit boards, cosmetic instruments, food preparation instruments, and other items in which reliable cleaning and sterility are desirable or required.

### EXAMPLE 1

The purpose of this test is to document the results of engineering characterization testing performed on a automatic endoscope reprocessor, the Langford I.C. Systems Sterilizer Cleaner (see U.S. Pat. No. 5,906,802 for layout and guidance in the use of this reprocessor). Testing was performed on DWGX-0129-01888, Cleaner, Sterilizer Breadboard.

The biopsy lumen of three colonoscopes were loaded with Hucker's Soil (much more than required by FDA test standards) and inoculated with pathogens from an American Society of Test Methods kit. The scopes were left sitting for a 24 hour time period to permit some drying. Using the same Langford I.C. Systems Sterilizer Cleaner liquid-displacement settings as described, each colonoscope was subjected to one wash cycle at 4 psi for 5 min with a 50%/50% (vol/vol) mixture of SIMPLE GREEN cleaner and peracetic acid sterilant in 10 liters of water. The preferred rate of “liquid displacement” (i.e., the back-and-forth liquid cycling rate in the item-washing chamber of the Sterilizer Cleaner) is 1 gallon per 2 seconds.

Upon completion of the sterilization cycle, the tray upon which the colonoscope is positioned is automatically raised and tilted such that the scope slides into a waiting sterile bag. The opening in the bag is then heat sealed by application of a heating element. Alternatively, the colonoscope goes through the cleaning step and the reprocessor is opened so the scope can be removed. A user then takes sterilizable packaging, such as a polyethylene sleeve, from a dispenser conveniently coupled to the push/pull apparatus, and places the scope inside. After replacing the packaged scope into the reprocessor, the sterilization cycle takes place by surging a liquid sterilant back-and-forth upon the colonoscope and open packaging. The packaging is then sealed shut with a heating element as above.

Tests carried out using “simulated soil” (e.g., Hucker's soil) show that reprocessing an instrument according to the present invention results in less than four micrograms per cm² of residual protein. In other words, any protein staying on the instrument from the simulated soil and/or pathogens is in an amount of less than 4 micrograms per cm² of surface area.

### EXAMPLE 2

In this example, a cleaning/sterilizing device of the type described and illustrated in U.S. Pat. No. 5,711,921 is utilized. A dental instrument is positioned to extend through the baffle of the device such that one opening of the endoscope lies in one chamber and another opening of the endoscope lies in the other chamber. The device creates this “surge” through the use of one or more flexible membranes. By deforming the flexible membrane (inward and outward), a pressure or suction is created which results in a flow (liquid displacement) between the chambers to equalize the pressure between them.

Next, 250 ml of detergent is added to 10 liters of water and is used to wash the instrument for 10 min. After 5 minutes of the first cycle, 10 liters of a liquid chemical sterilant (preferably 1 ounce peracetic acid per 5 liters of water) are added to the cleaner/sterilizer and the endoscope continues to be treated for 5 minutes. Those of ordinary skill in the art readily recognize various other sterilants which can be used in this context.

The dental instrument then goes through a drying cycle, whereupon a sterile sleeve is retrieved from the dispenser by a user of the reprocessor and held at an end of the instrument such that the instrument can be easily slid into the sleeve and sealed.

### EXAMPLE 3

As used in this example, the term “plasma” is intended to include any portion of the gas or vapors which contains electrons, ions, free radicals, and the like produced as a result of an applied electrical field, including any accompanying radiation that might be produced. While radiation in the radio frequency range is most commonly applied, a broad frequency range may be used.

A medical item is placed in a multi-chamber reprocessor and cleaned as above. The item is then packaged and sealed in a coated paper envelope and transferred to a chamber separate from the cleaning chamber. The separate chamber includes a fluid port to inject gas into the chamber, along with an aqueous solution of hydrogen peroxide into the chamber, and a vacuum line to evacuate the chamber. The separate chamber includes radio frequency electrodes to generate the requisite radio frequency signal. The plasma is generated by evacuating the chamber, introducing a gas or vaporized liquid and turning on the power to the electrodes. The plasma is generated in the present process in the same manner as in known prior art plasma sterilization system (e.g., U.S. Pat. No. 4,643,876).

The hydrogen peroxide is injected in the form of an aqueous solution of hydrogen peroxide containing from about 3% to 20% by weight of hydrogen peroxide. The concentration of hydrogen peroxide vapor in the chamber may range from 0.05 to 10 mg of hydrogen peroxide per liter of chamber volume. A concentration of 0.125 mg per liter is the minimum preferred concentration of hydrogen peroxide. Air or an inert gas such as argon, helium, nitrogen, neon or xenon may be added to the chamber with the hydrogen peroxide to maintain the pressure in the chamber at the desired level. The hydrogen peroxide solution may be injected in one or more separate injections.

Apparently, the hydrogen peroxide diffuses through the packaging material and comes into close proximity, if not contact, with the surface of the item to be sterilized. Upon the application of power to the radio frequency generator, sporadically active species are generated by the combination of hydrogen peroxide and plasma.

The general operation of the present process utilizing plasma may be thus be summarized as follows: (1) The object or article to be sterilized is placed in an all-in-one chamber or into the plasma chamber; (2) The chamber is evacuated to a pressure of approximately 0.05 Torr; (3) An aqueous solution of hydrogen peroxide is injected into the chamber to a pressure of vaporized water and hydrogen peroxide of from 0.5 to 10 Torr (the preferred pressure is from 1 to 2 Torr and the concentration of the hydrogen peroxide injected into the chamber may be from about 0.05 to 10 mg/liter of chamber volume); (4) The object to be sterilized is held in the chamber before plasma with sufficient power to sterilize is generated for a period of from about 5 to 30 minutes; (5) The object to be sterilized is subjected to a plasma either in the pre-treatment chamber or in a separate plasma chamber; (6) The RF energy used to generate the plasma may be continuous or it may be pulsed. The object remains in the plasma for a period of from 5 to 60 minutes to effect complete sterilization.

Since the hydrogen peroxide is decomposed into non-toxic products during the plasma treatment, no additional steps are required to remove residual hydrogen peroxide from the sterilized object or its packaging prior to use of the object.

Alternatively, if sterilization of a lumened instrument (e.g., a bronchoscope) is desired, the above method would be modified in the push/pull reprocessor to surge gas plasma back-and-forth upon all accessible surfaces, including the exterior and through the lumen of the scope, before any packaging would take place. Surging would be employed because gas plasma (or other vapor-phase sterilants, such as steam) cannot effectively permeate through the lumen.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products. All references cited in this application are hereby incorporated by reference herein.

What is claimed is:

1. A method for reprocessing and packaging items, comprising the steps of:
a. cleaning an item within a push/pull reprocessing apparatus;
b. containing said item within sterilizable packaging; and
c. sterilizing said contained item within said apparatus.

2. The method of claim 1, wherein said packaging comprises a disposable sleeve.

3. The method of claim 2, wherein said sleeve comprises polyethylene with sealable ends.

4. The method of claim 1, wherein said step (c) comprises surging a sterilant back-and-forth upon said item and container.

5. The method of claim 1, wherein said step (c) comprises passive exposure to a sterilant.

6. The method of claim 1, wherein said sterilant is selected from the group consisting of a liquid or a gas.

7. The method of claim 1, further including the step of drying said item prior to said step of containing.

8. A method for reprocessing and packaging items, comprising the steps of:
a. cleaning an item within a push/pull reprocessing apparatus;
b. removing said item from said apparatus;
c. containing said item within sterilizable packaging;
d. replacing the contained item into the apparatus; and
e. sterilizing said contained item within said apparatus.

9. The method of claim 8, wherein said packaging comprises a disposable sleeve.

10. The method of claim 9, wherein said sleeve comprises polyethylene with sealable ends.

11. The method of claim 8, wherein said step (e) comprises surging a sterilant back-and-forth upon said item and container.

12. The method of claim 8, wherein said step (e) comprises passive exposure to a sterilant.

13. The method of claim 8, wherein said sterilant is selected from the group consisting of a liquid or a gas.

14. The method of claim 8, further including the step of drying said item prior to said step of containing.

15. An apparatus for reprocessing and packaging an item, comprising:
a push/pull reprocessing apparatus; and
a dispenser of sterilizable packaging coupled to said push/pull reprocessor apparatus.

16. The apparatus of claim 15, wherein said dispenser is disposed upon the interior of said reprocessing apparatus.

17. The apparatus of claim 15, wherein said push/pull reprocessing apparatus contains a single chamber within which both cleaning and sterilization after packaging takes place.

18. The apparatus of claim 15, wherein said push/pull reprocessing apparatus includes a cleaning chamber and a separate sterilization-after-packaging chamber.

19. The apparatus of claim 15, wherein said push/pull reprocessing apparatus includes a cleaning chamber and a separate sterilization-after-packaging chamber, wherein said separate chamber is connected to a fluid port that dispenses sterilant.

20. The apparatus of claim 19, wherein said fluid port dispenses gas plasma.

21. A method for reprocessing and packaging items, comprising the steps of:
a. cleaning an item within a push/pull reprocessing apparatus;
b. sterilizing said item within said apparatus; and
c. containing said item within sterile packaging.

22. The method of claim 21, wherein step (b) is performed with gas plasma.

23. The method of claim 21, wherein step (a) results in less than 4 micrograms per centimeter squared of residual protein.

24. The method of claim 21, wherein step (a) results in less than 4 micrograms per centimeter squared of residual protein from a simulated soil.

25. The method of claim 21, wherein (b) comprises a final rinse with sterile water that contains particles fewer than 10 micrometers in size.

26. The method of claim 25, wherein said water contains particles 0.1 micrometers in size or less.

27. A method for reprocessing an item, comprising the steps of:
a. cleaning the item within a push/pull reprocessing apparatus; and
b. sterilizing the item within said apparatus by surging gas plasma back-and-forth upon a surface of said item prior to any packaging of said item.
